(19) United States
(12) Patent Application Publication
(10) Pub. No.: US 2022/0378308 A1
(43) Pub. Date: Dec. 1, 2022

(54) Method And Device That Monitors A Fetal Heart Rate

(71) Applicants: Zepp, Inc., Cupertino, CA (US); Anhui Huami Health Technology Co., Ltd., Hefei (CN)

(72) Inventors: Artem Galeev, Vancouver (CA); Vahid Zakeri, Vancouver (CA); Yan Vule, Port Moody (CA); Kongqiao Wang, Hefei (CN)

(21) Appl. No.: 17/332,126

(22) Filed: May 27, 2021

(51) Int. Cl. A61B 5/024; A61B 5/318; A61B 5/00; A61B 5/113

(52) U.S. Cl. CPC A61B 5/02411; A61B 5/318; A61B 5/743; A61B 5/7207; A61B 5/681; A61B 5/02416; A61B 5/002; A61B 5/113; A61B 2562/0219

(57) ABSTRACT

A device comprising: (a) an accelerometer or gyroscope that is capable of measuring movement, and (b) a sensor that is configured to monitor and determine a electrical signals or pulse signals of a heart so that the sensor detects a heart rate of a user; wherein the device is configured for placement on a first location of the user to determine a heart rate of the user; wherein the device is configured for placement on or contact with a second location of the user where the sensor measures the user's heart rate and the accelerometer or gyroscope measures a heart rate of a fetus located within the user; and (c) a processor configured to isolate the heart rate of the fetus from the heart rate of the user so that the heart rate of the fetus is displayed on the device.

### CROSS REFERENCES TO RELATED APPLICATION(S)

None.

### FIELD

The teachings herein relate to a device and method of measuring a fetal heart rate and more specifically isolating a fetal heart rate from a heart rate of an individual carrying the fetus.

### BACKGROUND

Many portable devices have been developed in which some sensors can be used to monitor various physical conditions of the user. Some of the physical conditions that may be measured include monitoring of heart rate, glucose level, apnea, respiratory stress, movement of the user, and other physiological conditions. While these device are good at monitoring physical conditions of the wearer or user these devices have been limited to only monitoring the user or wearer.

It would be attractive to have a device that allows a pregnant user to monitor one or more conditions of their fetus. What is needed is a device that is configured to monitor a fetus. It would be attractive to have a device, method, or both that monitors a status of one or more physical conditions of a fetus and can isolate the one or more physical conditions of the fetus from the device user or mother. What is needed is a device that is capable of monitoring a fetus in utero and providing feedback to the user, wearer, or mother regarding physical conditions of the fetus.

### SUMMARY

Disclosed herein are implementations of a wearable device for measuring heart rates and specifically a heart rate of a user and a fetus.

The present teachings provide: a device comprising: (a) an accelerometer and/or gyroscope that is capable of measuring movement, and (b) a sensor that is configured to monitor and determine electrical signals or pulse signals of a heart so that the sensor detects a heart rate of a user; wherein the device is configured for placement on a first location of the user to determine a heart rate of the user; wherein the device is configured for placement on or contact with a second location of the user where the sensor measures the user's heart rate and the accelerometer or gyroscope measures a heart rate of a fetus located within the user; and (c) a processor configured to isolate the heart rate of the fetus from the heart rate of the user so that the heart rate of the fetus is displayed on the device.

The present teachings provide: a device comprising: a non-transitory computer-readable medium configured to: monitor acceleration, velocity, or both information from an accelerometer and/or gyroscope; monitor a electrical signals or pulse signals of a heart with a sensor; determine a heart rate of a user of the device; determine a heart rate of a fetus of the user with acceleration and/or velocity information from the accelerometer or gyroscope; determine the heart rate of the fetus by removing the heart rate of the user from data collected so that the heart rate of the fetus is an only remaining data; and displaying the heart rate of the fetus.

The present teachings provide: a method comprising: (a) monitoring an accelerometer or gyroscope to collect acceleration and/or velocity information; (b) correlating the acceleration and/or velocity information to create a first data set related to a heart rate of a fetus of a user; (c) monitoring a sensor to determine electrical signals or pulse signals of a heart to create a second data set correlated to the user's heart rate; and (d) removing the second data set from the first data set so that the user's heat rate is removed from the heart rate of the fetus.

The present teachings provide a device that allows a pregnant user to monitor one or more conditions of their fetus. The present teachings provide a device that is configured to monitor a fetus. The present teachings provide a device, method, or both that monitors a status of one or more physical conditions of a fetus and can isolate the one or more physical conditions of the fetus from the device user or mother. The present teachings provide a device that is capable of monitoring a fetus in utero and providing feedback to the user, wearer, or mother regarding physical conditions of the fetus.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 depicts a device according to the teachings herein.

FIG. 2 depicts a module of the device taught herein.

FIG. 3 depicts a side view of the device taught herein.

FIG. 4A depicts a device taught herein.

FIG. 4B depicts a first side the device taught herein being worn.

FIG. 4C depicts a second side the device taught herein being worn.

FIG. 5 is a front perspective view of a user with a device around a mid-section.

FIG. 6 illustrates the device with a band connected to an extended band.

FIG. 7 illustrates the device being used to sense a user heart rate and a fetal heart rate.

FIG. 8 illustrates a lower module removed from the device and connected to an extended band.

FIG. 9 illustrates an example of a fetal heart rate and user heart rate measured by a lower module located above a user heart rate measured by an upper module.

FIG. 10 is a flow chart showing steps to provide a fetal heart rate.

### DETAILED DESCRIPTION

Disclosed herein is a device that senses, measures, analyzes, and/or displays physiological information. In one aspect, the device may be a wearable device comprising an upper module and/or a lower module. The wearable device may be worn on a user's body such that one or more sensors of the upper and lower modules contact a targeted area of tissue. In one implementation, the wearable device is a watch, band, or strap that can be worn on the wrist of a user such that the upper and lower modules are each in contact with a side of the wrist.

The present teachings provide a wearable device that functions to monitor a heart rate of a user and a heart rate of a fetus being carried by the user (i.e., inside the user's body). The wearable device may monitor physiological information (e.g., a heart rate, respiration) of a user at a first location. The wearable device may monitor a first location and a second location simultaneously for physiological information. For example, the wearable device may monitor a wrist (e.g., first location) of a user and a mid-section (e.g., second location) of a user. The wearable device may simultaneously measure a heart rate of a fetus and a heart rate of a user. The wearable device may be placed at or in contact with a first location, a second location, or both in series or in parallel. For example, the wearable device may be strapped around a wrist, mid-section, or both. The wearable device may be placed proximate to a location or in contact with a location. For example, while strapped to a wrist the wearable device may be placed on a mid-section of the user so that accelerations or velocities within the mid-section of the user are measured. The physiological information measured at the first location and the second location may be the same physiological information or may be a different physiological information. For example, the wearable device may be located on a wrist of a user (e.g., first location) and may be placed proximate to or in contact with an abdomen of a user (e.g., second location). The wearable device may be connected to a wrist and may be held against a midsection (e.g., abdomen) so that physiological information of a user and a fetus within the user may be measured simultaneously. The wearable device may be connected to a wrist and a portion of the device may be moved from the wrist into contact with the mid-section. The wearable device may be moved to a second location where the wearable device may monitor the fetus's heart rate and the user's heart rate. For example, all or a portion of the wearable device may be attached around a mid-section of the user so that the wearable device may monitor a fetus in utero. The wearable device may include one or more modules and the modules may be individually moved so that two separate locations may be monitored at the same time. For example, a first module may monitor at a first location and a second module may monitor at a second location. The wearable device may function to isolate physiological information of a user from physiological information of a fetus or vice versa. The physiological information may be a heart rate. The wearable device may function to isolate the fetus's heart rate, filter out the user's heart rate, remove the user's heart rate, or a combination thereof so that the fetus's heart rate, the user's heart rate, or both may be displayed. The wearable device may include one or more sensors or one or more modules. The wearable device may include a plurality of modules that each include one or more sensors. The wearable device may include an upper module, a lower module, or both.

The present teachings provide a wearable device that may include a lower module that may be attached to another device. For example, the wearable device may include a lower module that is a clip and/or an add-on to a watch or another wearable device. For example, the lower module may be attachable to the bottom of a watch such that the lower module is in contact with the skin of the wearer. The lower module may be removable from the wearable device and may maintain communication (e.g., wireless communication) with the wearable device. The lower module may wirelessly communicate with the wearable device. The lower module may include memory that stores measurements and once reconnected the lower module synchs with the portable device. The lower module may include an accelerometer, gyroscope, or both. The accelerometer or gyroscope functions to measure movement of a user. The accelerometer or gyroscope may measure micro-movements, macro-movements, or both. The lower module may be removable from a first location (e.g., a wrist) and placed at or on a second location (e.g., mid-section or abdomen). The lower module may be connectable to a belt or piece of clothing. The lower module may be held in place by a user's hand. The lower module may be placed directly into contact with the mid-section or abdomen so that movements within the user may be detected (e.g., movements caused by a heartbeat of a fetus). The lower module may include one or more of the sensors discussed herein. The lower module may include only an accelerometer or gyroscope. The lower module may monitor micro-movements, acceleration, velocity changes, or a combination thereof. The lower module and the upper module may include some of the same sensors. The lower module may face outward relative to the user. The lower module may be located on an opposite side of a band as an upper module. The lower module and the upper module may include some different sensors.

The upper module may include one or more sensors. The sensors may monitor electrical signals of the heart. The heart may receive electrical signals that cause the heart to beat and the sensors may monitor the electrical signals. The sensors may monitor pulse signals. The pules signals may be movement of a part of the body caused by blood being moved through the body. The pulse signals may be movement of blood through a vein or artery. The pulse signals may monitor expansion and contraction of veins and arteries using a light. The upper module may include an optical sensor (e.g., PPG), a pulse pressure sensor (PP), a pressure sensor, an electrocardiogram (ECG), or a combination thereof. The sensors may be any device that may measure a heart rate. The optical sensors may provide a heart rate via a PPG. The sensor may provide a heart rate via a pressure sensor (PP) or ECG. The upper module may be removable from the movable device. The upper module may be fixed within the removable device. The upper module may be located opposite the lower module (e.g., 180 degrees apart).

Each of the upper and lower modules may comprise one or more sensors, including but not limited to optical sensors (e.g., PPG), Electrocardiogram (ECG) sensors/electrodes, bio impedance sensors, galvanic skin response sensors, tonometry/contact sensors, accelerometers, gyroscopes, pressure sensors, acoustic sensors, electro-mechanical movement sensors, and/or electromagnetic sensors. The one or more optical sensors (e.g., PPG) may comprise one or more light sources for emitting light proximate a targeted area of tissue and one or more optical detectors for detecting either reflected light (where an optical detector is located on the same side of the targeted area as the light source(s), i.e., within the same module) or transmitted light (where an optical detector is located opposite the light source(s), i.e., within an opposing module). The optical sensor may be a light emitting diode and photodiode (e.g., LED+photodiode) to measure PPG. The pressure sensor may monitor pulse pressure to provide a PP reading.

The strap or band of the wearable device may be configured so as to facilitate proper placement of one or more sensors of the upper and/or lower modules while still affording the user a degree of comfort in wearing the device. The present teachings may provide a strap that lies in a plane perpendicular to the longitudinal axis of the user's wrist or arm (as is the case with traditional wrist watches and fitness bands), the band may be configured to traverse the user's wrist or arm at an angle that brings one or more components of the upper or lower modules into contact with a specific targeted area of the user while allowing another portion of the band to rest at a position on the user's wrist or arm that the user finds comfortable, or around a mid-section of the user. The strap or band may be expandable so that the band is movable from a wrist to a mid-section. The strap or band may be connected to another strap or band so that the wearable device may be moved from a wrist to a mid-section. For example, the wearable device may be connected to a belt extending around a mid-section of the user. The strap or band may be expandable to accommodate different body parts of a user (e.g., a wrist or a mid-section). The strap or band may be fitted around a mid-section and then a user places their wrist over the wearable device to measure the user's physiological information from their wrist while physiological information is being measured from the mid-section. For example, a predetermined amount of pressure may be desired to hold the wearable device on the mid-section or abdomen of the user and the strap or band may assist in creating the predetermined amount of pressure so that an accurate reading is taken. The precise location of the upper and/or lower modules can be customized such that one or more sensors of either module can be placed in an ideal location of a user, despite the physiological differences between body types from user to user.

The physiological information sensed, measured, analyzed, or displayed can include but is not limited to heart rate information, ECG waveforms, calorie expenditure, step count, speed, blood pressure, oxygen levels, pulse signal features, cardiac output, stroke volume, and respiration rate of a user, breathing movements of a fetus, heart rate of a user, heart rate of a fetus, or a combination thereof. The physiological information may be any information associated with a physiological parameter derived from information received by one or more sensors of the wearable device. The physiological information may be used in the context of, for example, health and wellness monitoring, athletic training, physical rehabilitation, patient monitoring, fetal monitoring, or a combination thereof.

The portable device may be a wearable device (such as a wrist-worn portable device) or a hand-portable device, that can be used by a user to measure electrical activity of the heartbeat of the user. The portable device may include an ECG sensor (e.g., an electrode) that the user may place on his/her chest or other body locations. The portable device (using a processor therein) may detect (e.g., determine, calculate, etc.) the location of the sensor on the body to determine a lead (e.g., an angle of measurement). The location of the sensor may be determined using sensors of a device that is external to the portable device. In an example, a digital image processing can be used. For example, an external camera that can be used to image the chest of the user as the portable device is placed on the chest. The location of the portable device can be determined using an image of the camera.

The portable device can be a wearable device that includes a strap and that can be worn on a wrist of a first arm of the user. A second sensor (e.g., an upper module or a lower module) can be included in the strap (e.g., a tail of the strap). While taking measurements, the user can touch the second sensor or hold the second sensor (such as between the thumb and index fingers of the other hand of the user or against a user's mid-section or abdomen). As such, the measurement can be more accurate when more electrodes (e.g., ECG sensors) are used. Alternatively, multiple measurements may be made of the user, a fetus within the user, or both. The second sensor may be held by the user to determine the user's physiological information while the device is monitoring a fetus within the user with a first sensor. The second sensor data may be used to determine which physiological information detected by the first sensor is that of the user and which physiological information is that of the fetus (e.g., one set of data may be reference data or data used for discrimination).

While the systems and devices described herein may be depicted as wrist worn devices, one skilled in the art will appreciate that the systems and methods described below can be implemented in other contexts, including the sensing, measuring, analyzing, and display of physiological data gathered from a device worn at any suitable portion of a user's body, including but not limited to, other portions of the arm, other extremities, the head, the chest, the abdomen or mid-section, or a combination thereof.

The wearable device functions to be configured to provide information about a fetus within the user. The wearable device functions to monitor a heart rate of a fetus being carried by the user. The wearable device may use a first sensor to monitor a user and a second sensor to monitor a fetus within the user. The first sensor may provide a first set of data and the second sensor may provide a second set of data. The first set of data may be removed from the second set of data. The second set of data may be filtered. The second set of data may have outliers removed. The second set of data may be filtered into micro-movements and macro-movements. The macro-movements may be categorized as an outlier, a macro-movement, or both. The micro-movements may include data from the user, the fetus, or both. The micro-movements may be filtered to remove user data from the fetus data so that only fetus data remains. The data measured by the second sensor may be acceleration data via an accelerometer or velocity data via a gyroscope. The data may be filtered via synchrosqueezing.

Synchrosqueezing functions to decompose data into a plurality of constituents. Sunchrosqueezing may apply an algorithm to the data so that the time-frequency representations of the data may be broken into basic constituents. For example, if the sensor monitors accelerations or velocities that include micro-movements, the sensor may also measure the accelerations or velocities of the user that may be indicative of other micro-movements or some macro-movements (e.g., heartbeats of a fetus within the user). The synchrosqueezing may separate out a first set of micro-movements (e.g., from fetus) from a second set of micro-movements (e.g., from a user) so that the first set or the second set may be isolated relative to one another. The synchrosqueezing may categorize the data based upon a frequency, an amplitude, frequency within a pre-determined amplitude, or a combination thereof. The synchrosqueezing may compare a second set of data to a first set of data. The synchrosqueezing may remove or filter out a constituent from a first data set with an identical or substantially similar constituent from a second data set. The synchrosqueezing may break apart a data set into one or more oscillating signals, two or more oscillating signals, three or more oscillating signals, intermittent signals, period signals, or a combination thereof. For example, one sensor may measure a user heart rate and a second sensor may measure accelerations and/or velocities that include breathing of the user, heartbeats of the user, movements of the user, movements of a fetus, heart rate of the fetus, or a combination thereof. The heart rate of the fetus may be a single isolated constituent that remains after the data is filtered. The data may be filtered with or without synchrosqueezing.

The processor functions to analyze acceleration data, velocity data, or both and to remove or isolate some of the constituents from the acceleration data, velocity data, or both based upon a separate measurement. The processor may subtract, remove, isolate, or a combination thereof the first measurement from the second measurement. For example, the first measurement may be a heart rate of a user and the second measurement may include a heart rate of a user and fetus and the heart rate of the user may be “removed” from the data so that all that remains is a heart rate of the fetus. The heart rates may be derived from movements (e.g., micro-movements) of a body part of a user (e.g., an abdomen or mid-section).

Reference will now be made in detail to certain illustrative implementations, examples of which are illustrated in the accompanying drawings. Wherever possible, the same reference numbers will be used throughout the drawings to refer to the same or like items.

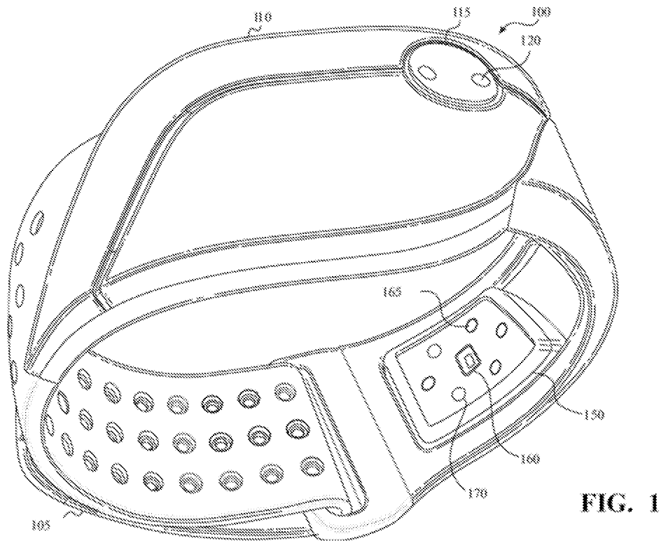

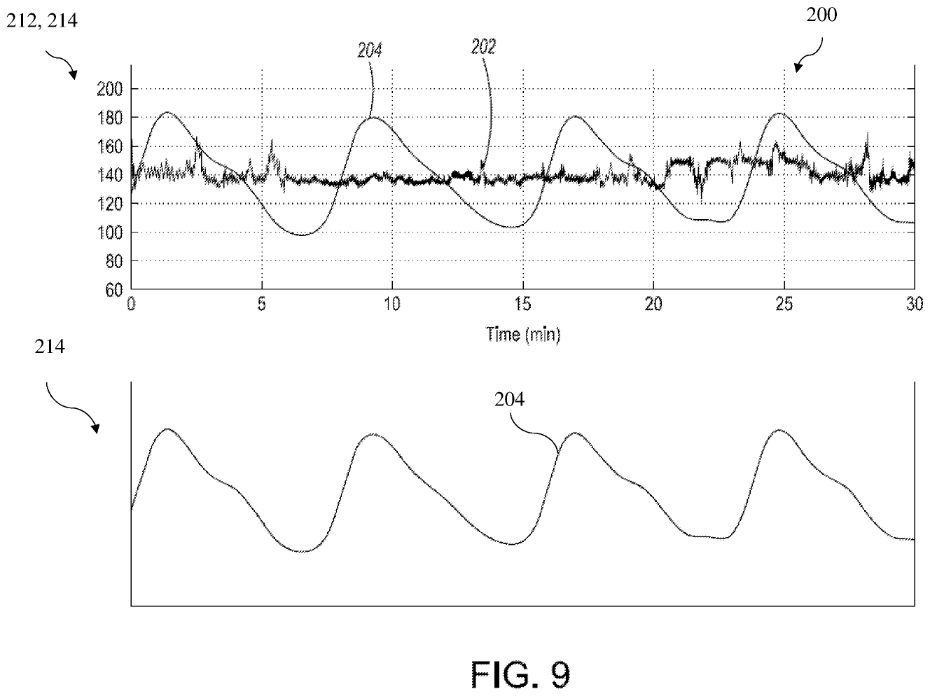

FIGS. 1-8 illustrate the device 100 and various examples of the device 100 during use. FIG. 9 is one representative illustration of an output 200 provided by the device. FIG. 1 depicts a perspective view of a device 100. The device 100 may be a physiological monitor worn by a user to sense, collect, monitor, analyze, and/or display information pertaining to one or more physiological parameters. The device 100 comprises a band, strap, or wristwatch. The device 100 is a wearable monitor device configured for positioning at a user's wrist, arm, another extremity of the user, or some other area of the user's body.

The device 100 may comprise at least one of an upper module 110 or a lower module 150, each comprising one or more components and/or sensors for detecting, collecting, processing, and displaying one or more physiological parameters of a user and/or other information that may or may not be related to health, wellness, exercise, or physical training sessions.

The upper module 110 and lower module 150 of the device 100 may comprise a strap or band 105 extending from opposite edges of each module for securing device 100 to the user. The band(s) 105 may comprise an elastomeric material or the band(s) 105 may comprise some other suitable material, including but not limited to, a fabric or metal material.

Upper module 110 or lower module 150 may also comprise a display unit (not shown) for communicating information to the user. The display unit may be an LED indicator comprising a plurality of LEDs, each a different color. The LED indicator can be configured to illuminate in different colors depending on the information being conveyed. For example, where device 100 is configured to monitor the user's heart rate, the display unit may illuminate light of a first color when the user's heart rate is in a first numerical range, illuminate light of a second color when the user's heart rate is in a second numerical range, and illuminate light of a third color when the user's heart rate is in a third numerical range. In this manner, a user may be able to detect his or her approximate heart rate at a glance, even when numerical heart rate information is not displayed at the display unit, and/or the user only sees device 100 through the user's peripheral vision.

The display unit may comprise a display screen for displaying images or characters to the user. The display unit may further comprise one or more hard or soft buttons or switches configured to accept input by the user. The display unit may switch or be toggled between displaying user physiological information or fetus physiological information.

Device 100 may further comprise one or more communication modules. Each of the upper module 110 and the lower module 150 may comprise a communication module such that information received at either module can be shared with the other module.

One or more communication modules may also communicate with other devices such as the user's cell phone, tablet, or computer. Communications between the upper and lower modules can be transmitted from one module to the other wirelessly (e.g., via Bluetooth, RF signal, Wi-Fi, near field communications, etc.) or through one or more electrical connections embedded in band 105. Any analog information collected or analyzed by either module can be translated to digital information for reducing the size of information transfers between modules. Similarly, communications between either module and another user device can be transmitted wirelessly or through a wired connection, and translated from analog to digital information to reduce the size of data transmissions.

As shown in FIG. 1, lower module 150 can comprise an array of sensor array 155 including but not limited to one or more optical detectors 160, one or more light sources 165, and one or more contact pressure/tonometry sensors 170. These sensors are only illustrative of the possibilities, however, and lower module may comprise additional or alternative sensors such as one or more acoustic sensors, electromagnetic sensors, ECG electrodes, bio impedance sensors, galvanic skin response sensors, gyroscopes, and/or accelerometers. Though not depicted in the view shown in FIG. 1, upper module 110 may also comprise one or more such sensors and components on its inside surface, i.e. the surface in contact with the user's tissue or targeted area.

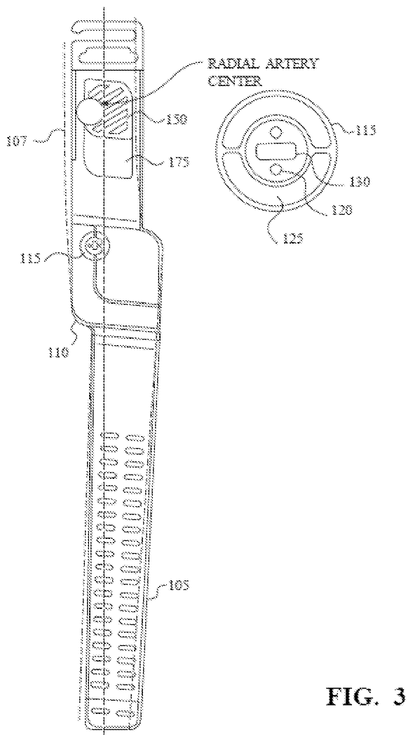

The location of sensor array 155 or the location of one or more sensor components of sensor array 155 with respect to the user's tissue may be customized to account for differences in body type across a group of users or placement in different locations on a user. For example, band 105 may comprise an aperture or channel 175 within which lower module 150 is movably retained. In one implementation, lower module 150 and channel 175 can be configured to allow lower module 150 to slide along the length of channel 175 using, for example, a ridge and groove interface between the two components (see e.g., FIG. 3). For example, if the user desires to place one more components of sensor array 155 at a particular location on his or her wrist, or mid-section, the lower module 150 can be slid into the desired location along band 105. Though not depicted in FIG. 1, band 105 and upper module 110 can be similarly configured to allow for flexible or customized placement of one or more sensor components of upper module 110 with respect to the user's wrist or targeted tissue area.

The sensors and components proximate or in contact with the user's tissue, upper module 110 and/or lower module 150 may comprise additional sensors or components on their respective outer surfaces, i.e. the surfaces facing outward or away from the user's tissue. In the implementation depicted in FIG. 1, upper module 110 comprises one such outward facing sensor array 115. The sensor array 115 may comprise one or more ECG electrodes 120. Similar to the sensor arrays of the upper and lower modules proximate or in contact with the user's tissue, outward facing sensor array 115 may further comprise one or more contact pressure/tonometry sensors, photo detectors, light sources, acoustic sensors, electromagnetic sensors, bio impedance sensors, galvanic skin response sensors, gyroscopes, and/or accelerometers.

The outward facing sensors of sensor array 115 can be configured for activation when touched by the user (with his or her other hand) and used to collect additional information. The outward facing sensors may measure without being in direct contact with the user. The outward facing sensors may be an accelerometer and the accelerometer may indirectly monitor movements or micro-movements that are transmitted to the sensor through the band or the module moving or being moved or a gyroscope that monitors velocities to determine micro-movements. In an example, where lower module 150 comprises one or more optical detectors 160 and light sources 165 for collecting ECG, PPG, or heart rate information of the user, outward facing sensor array 115 of upper module 110 may comprise ECG electrodes 120 that can be activated when the user places a fingertip in contact with the electrodes. While the optical detectors 160 and light sources 165 of lower module 150 can be used to continuously monitor blood flow of the user, outward facing sensor array 115 of upper module 110 can be used periodically or intermittently to collect potentially more accurate blood flow information which can be used to supplement or calibrate the measurements collected and analyzed by an inward facing sensor array, the sensor array 155, of lower module 150.

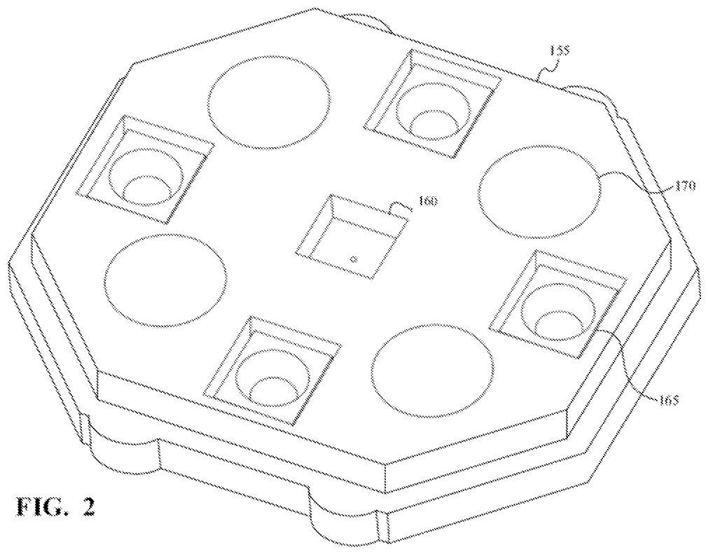

FIG. 2 depicts an inward facing sensor array, the sensor array 155, of lower module 150 according to the teachings herein. The sensor array 155 can comprise sensors including but not limited to one or more optical detectors 160, one or more light sources 165, one or more contact pressure/tonometry sensors 170, and an accelerometer (not shown). The sensor array 155 may comprise additional or alternative sensors such as one or more acoustic sensors, electromagnetic sensors, ECG electrodes, bio impedance sensors, galvanic skin response sensors, and/or accelerometers. Upper module 110 may comprise a similar inward facing sensor array (not depicted in FIG. 1) configured to position sensors proximate or in contact with the outside portion of a user's wrist or arm. Sensor components of the upper module 110 and the lower module 150 can be used in combination to collect and analyze physiological information. For example, one or more light sources of lower module 150 can be used to transmit light through a targeted area of the user's tissue (e.g., a portion of the user's wrist) and the transmitted light can be detected by one or more photodetectors of an inward facing sensor array of upper module 110. Opposing modules 110 and 150 can be used to detect and analyze either reflected or transmitted light.

FIG. 3 depicts a side view of the device 100 comprising a band 105, upper module 110, and lower module 150 according to the teachings herein. The lower module 150 can be placed within channel 175 of band 105 such that lower module 150 can slide along the longitudinal axis of band 105. The movability of lower module 150 (or upper module 110 in alternative implementations) with respect to band 105 allows a user to customize the location of the inward facing sensors of lower module 150 with respect to a targeted tissue area to ensure reliable and accurate detection of physiological parameters. For example, a user can ensure that the inward facing sensors of lower module 150 are place in a location proximate the center of the user's radial artery.

The band 105 may not extend around the user's wrist such that the band 105 traverses a circumferential path lying in a plane perpendicular to the longitudinal axis of the user's wrist or arm. The longitudinal axis of band 105 extends at an angle such that portions of inward facing sensor arrays of upper or lower modules 110, 150 can be placed at suitable locations proximate a desired targeted area of tissue while a portion of band 105 is in contact with portions of the user's wrist or arm that the user finds comfortable (i.e., above or below the wrist joint). In some implementations, where a circumferential path around a user's wrist resides in a plane perpendicular to the longitudinal extension of the user's arm or wrist, band 105 may be set at an angle 107 with respect to the perpendicular plane. In some implementations, angle 107 may be between 5° and 15° with respect to the perpendicular plane. In other implementations, angle 107 may be less than 5° or more than 15°. Of primary importance is the placement of one or more components of the sensor arrays of upper and lower modules 110, 150 proximate or in contact with a desired targeted area of tissue while allowing a portion of band 105 to be worn at a comfortable location off the user's wrist joint. Additional details regarding proper or desirable placement of one or more sensors with respect to targeted tissue areas of a user are described below with respect to other figures.

FIG. 3 also shows a closer view of outward facing sensor array 115. In the implementation depicted, sensor array 115 may comprise one or more ECG electrodes 120 for establishing an electrical connection with a user's fingertip and collected ECG data. Sensor array 115 may further comprise one or more contact pressure/tonometry sensors 125 for detecting the presence of the user's fingertip, which can trigger activation of the ECG electrodes 120. Sensor array 115 may also comprise additional or alternative components 130 such as one or more optical detectors, light sources, acoustic sensors, electromagnetic sensors, bio impedance sensors, galvanic skin response sensors, and/or accelerometers.

The angle 107 of the band described with respect to FIG. 3 cures this deficiency in that it allows one or more sensor components of lower module 150 while allowing a portion of the remaining band and/or upper module 110 to be positioned at a more comfortable location on the user's wrist or arm.

The lower module 150 can slide along band 105. This allows the user to make further adjustments to the location of one or more sensors, not just along the longitudinal extension of the user's arm when device 100 is in use, but also along the circumferential extension of band 105 while device 100. Thus, the combination of band 105 extending around the user's wrist at an angle 107 together with the ability to slide the lower module 150 along band 105, ensure the sensors of lower module 150 can be placed at an ideal location with respect to each user (even users of different body types and physical attributes) and that the physiological parameters detected and analyzed by device 100 are collected as accurately as possible.

Device 100 is configured so as to ensure proper placement of one or more sensors and comfortability of band 105, but it also may contain additional sensors, such as a pressure sensor, at locations of device 100 other than upper and lower modules 110, 150.

The device 100 may comprise a pressure sensor located somewhere else along band 105 or at a latch that secures opposing ends of band 105 around a user's wrist for detecting pressure. Such a sensor can be used to ensure that the user is wearing the device 100 tightly enough to ensure one more other sensors are in sufficient contact with a targeted area of the user's tissue to collect accurate physiological information. One or more pressure sensors of the upper and/or lower modules 110, 150 may be used to collect accurate physiological information. In either case, device 100 may also be configured to alert the user (for example, via the display unit of upper module 110) if device 100 is being worn too loosely or too tightly to ensure accurate measurements.

Wrist worn ECG, PPG, PPM, or a combination thereof sensors may use a reflective system whereby a sensor array comprises one or more light sources and one or more optical detectors, located near one another and on the same side of a user's targeted area. The one or more light sources of the sensor array illuminate a portion of the user's tissue and light is reflected back to the optical detector(s) of the sensor array. The reflected light detected by the optical detector can be analyzed to estimate physiological parameters such as blood flow and pulse rate.

However, reflective systems may not be as accurate as transmissive systems that place one or more light sources on one side of a user's body and optical detectors on an opposing side of the user's body. A transmissive system may be a fingertip monitor used in a clinical setting. The monitors are clipped to a patient's fingertip, one side comprising a light source for illuminating the top or bottom of the patient's fingertip, the other side comprising an optical detector for detecting the light transmitted through the fingertip.

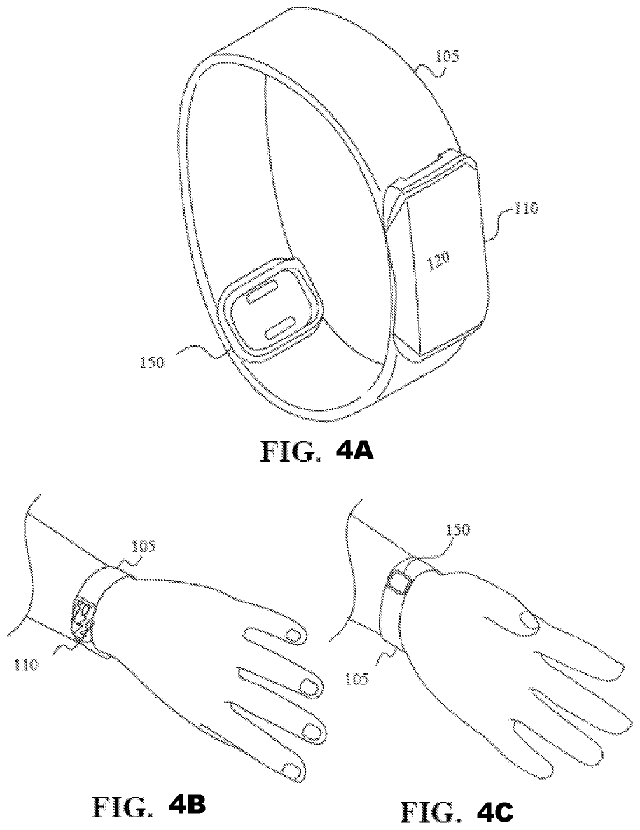

The device 100 may be located at the location of the radial artery (e.g., CUN artery) at the wrist at the inside of the wrist just under the thumb. As shown in FIGS. 4A, 4B, and 4C, device 100 can be configured to place the lower module 150 comprising a light source (and/or optical detector) at the location of the CUN artery on the underside of the wrist and place the upper module 110 comprising an opposing optical detector (or light source) at a location opposite the sensors of the lower module at the periphery of the outside of the wrist just below the thumb. In this manner, the path of light transmitted through the wrist between the sensors of the lower and upper modules travels a shorter distance than if the sensors were located closer to the center of the inside and outside of a user's wrist. As a result, light illuminated from either the upper or lower module can be detected at the opposing module in a manner previously only available in clinical settings and limited to locations on the body such as the fingertip.

The device 100 may comprise a number of components and sensors for detecting physiological information and extracting data from the device 100, such as blood flow, heart rate, fetal heart rate, respiratory rate, blood pressure, steps, calorie expenditure, and sleep patterns. Data collected from at least one or more of ECG electrodes/sensors, bio impedance sensors, galvanic skin response sensors, tonometry/contact sensors, accelerometers, gyroscopes, pressure sensors, acoustic sensors, and electromagnetic sensors can be used for determining physiological information.

One method for determining the heart rate, respiratory rate, blood pressure, oxygen levels, and other parameters of a user involves collecting a signal indicative of blood flow pulses from a targeted area of the user's tissue. As described above, this information can be collected using, for example, a light source, a photo detector, or a pressure sensor. Some implementations may use multiple light sources and they may be of varying colors (e.g., green, blue, red, etc.). For example, one light source may be an IR light source and another might be an LED light (such as a red LED). Using both an IR light source and a colored LED light (such as red) can improve accuracy as red light is visible and most effective for use on the surface of the skin while IR light is invisible yet effective for penetration into the skin. Such implementations may comprise multiple photo detectors, one or more configured to detect colored LED light (such as red) and one or more configured to detect IR light. These photo detectors (for detecting light of different wavelengths) can be combined into a single photodiode or maintained separate from one another. Further, the one or more light sources and one or more photodetectors could reside in the same module (upper or lower) in the case of a reflective system or the light source(s) could reside in one module while the optical detector(s) reside in the other in the case of a transmissive system.

Upon collection of a blood flow pulse signal, a number of parameters can be extracted from both single pulses and a waveform comprising multiple pulses. Features or parameters extracted from a single pulse can include, but are not limited to, shape of the pulse, a maximum amplitude, a minimum amplitude, a maximum derivative, a time difference between main and secondary peaks, and integral through the entire extraction time (i.e., the area under the pulse).

A PPG or photoplethysmogram system comprising one or more light sources and/or one or more optical detectors, can be supplemented with additional sensors such as ECG electrodes/sensors, bio impedance sensors, galvanic skin response sensors, tonometry/contact sensors, accelerometers, pressure sensors, acoustic sensors, and electromagnetic sensors. For example, one or more tonometry/contact sensors can be used to extract tonometry information by measuring the contact vessel pressure. Internal facing PPG components (i.e., one or more light sources and one or more photo detectors) may be used to detect reflected or transmitted light representative of blood flow pulses and some extrapolation of the data is made to determine, for example, heart rate, the user can place a fingertip of his or her off-hand on an outward facing ECG electrode (such as that shown in FIG. 1) to collect a more precise heart rate measurement. The more precise, though of more finite duration, heart rate measurement can be used to aid in the interpretation of the continuous heart rate measurements collected by the inward facing PPG sensors. The outward facing sensor can also comprise other sensors previously described herein, such as one or more contact/tonometry sensors, one or more bio impedance sensors, and one or more galvanic skin response sensors for analyzing electric pulse response. The outward facing sensors may monitor something other than the user. For example, the outward facing sensors may face away from the user and monitor a fetus within the user. All of the information collected by an outward facing sensor from, for example, the fingertip of the user's off-hand, can be used to refine the analysis of the continuous measurements taken by any one or more of the inward facing sensors. For example, an inward facing sensor may focus on monitoring fetal physiological information and the outward facing sensor may focus on monitoring user physiological information or vice versa.

In addition to the inward and outward facing sensors, device 100 may further comprise additional internal components such as one or more accelerometers and/or gyroscopic components for determining whether and to what extent the user is in motion (i.e., whether the user is walking, jogging, running, swimming, sitting, or sleeping). Information collected by the accelerometer(s) and/or gyroscopic components can also be used to calculate the number of steps a user has taken over a period of time. This activity information can also be used in conjunction with physiological information collected by other sensors (such as heart rate, respiration rate, blood pressure, etc.) to determine a user's caloric expenditure and other relevant information. The activity information may measure movements. The movements measured may be macro-movements such as walking or jogging. The movements may be micro-movements. The micro-movements may be caused by a surface of a user's skin being moved due to respiration, heartbeat, a fetus moving, a fetal heartbeat, fetal breathing movements (e.g., practice breathing movements of a fetus), or a combination thereof. The micro-movements may be movements of a user's mid-section. The micro-movements may have an amplitude (e.g., length) less than a predetermined amplitude in order for the accelerometer and/or gyroscope to measure and/or record the micro-movements. For example, when a user walks the accelerometer may measure a movement of more than 1 cm, when the accelerometer detects a user heart beat the accelerometer may measure a movement of between 4 mm and 1 cm, and when the accelerometer measures fetal heart rate the accelerometer may measure a movement of 4 mm or less (e.g., a micro-movement). The micro-movements may be charted in wave form such that the micro-movements are charted with a peak and a valley. The amplitude of movement may assist the non-transitory computer readable medium or processor in isolating movements caused by multiple sources (e.g., user or fetus).

To determine a user's blood pressure, the PPG information described above may be combined with other sensors and techniques described herein. Determining a user's blood pressure can comprise collecting a heart rate signal using a PPG system (i.e., one or more light sources and photo detectors) and performing feature extraction (described above) on single pulses and a series of pulses. The features extracted from single pulses and series of pulses can include statistical averages of various features across a series, information regarding the morphological shape of each pulse, the average and standard deviation of morphology of a series of pulses, temporal features such as the timing of various features within single pulses, the duration of a single pulse, as well as the average and standard deviation of the timing of a feature or duration of pulses within a series of pulses, and the timing of morphological features across a series of pulses (i.e., the frequency with which a particular pulse shape occurs in a series).

As described above, this feature extraction can not only be performed on a series of pulses and single pulses, but also on portions of a single pulse. In this manner, information pertaining to both systolic and diastolic blood pressure can be ascertained as one or more portions of an individual pulse correspond to the heart's diastole (relaxation) phase and one or more other portions of an individual pulse correspond to the heart's systole (contraction) phase. In some implementations, up to 200 features can be extracted from a partial pulse, a single pulse, and/or a series of pulses. The feature extraction may be applied to remove or isolate fetal physiological information from user physiological information.

In addition to features extracted from PPG or ECG information, information and features can also be collected by contract/tonometry sensors, pressure sensors, bio impedance sensors galvanic skin response sensors, accelerometers, acoustic sensors, and electromagnetic sensors. For example, pressure sensors or bio impedance sensors can be used to identify blood flow pulses of user and, similar to PPG or ECG data, features can be extracted from the collected data.

When the user's extracted features are compared to features recorded in the library, device 100 can also weigh the entries of subjects most closely corresponding to the user more heavily than entries of subjects associated with indicia different from that of the user. For example, if the user is a male, features extracted from male subjects may be weighed more heavily than female subjects because a particular pulse variation in men of a particular age may correspond to relatively high blood pressure whereas the same pulse variation in women of that particular age may correspond to lower blood pressure.

According to the techniques described herein, accurate blood pressure estimates for a user can be made without requiring direct blood pressure measurement of the user. However, in some implementations, the user's blood pressure estimates can be further calibrated by direct measurement of the user's blood pressure by another device and that verified blood pressure can be input into device 100 to aid in future estimations of the user's blood pressure. Calibration can also be accomplished with an outward facing ECG sensor.

While an inward facing PPG sensor can continuously or periodically collect heart rate data of a user, occasionally the user may be prompted to place a fingertip of his or her off-hand on an outward facing ECG sensor (e.g., electrodes). The inward facing sensor arrays of device 100 may contain additional electrodes thereby completing an electrical circuit through the user's body and allowing a more precise pulse waveform to be collected. Feature extraction can be performed on these pulses, series of pulses, and partial pulses in the same manner as described above with respect to PPG information and used to cross-reference the library.

Where device 100 determines, based on its continuous or periodic monitoring of the user's blood pressure using ECG, PPG, and/or pressure sensors, that a user's blood pressure is unusually or dangerously high or low, device 100 may prompt the user to place a fingertip of an off-hand on an outward facing ECG electrode in order to verify the unusual or unsafe condition. If necessary, device 100 can then alert the user to call for help or seek medical assistance.

As described above, the upper and/or lower modules 110, 150 can be configured to continuously collect data from a user using an inward facing sensor array. However, certain techniques can be employed to reduce power consumption and conserve battery life of device 100. For instance, only one of the upper or lower modules 110, 150 may continuously collect information. The module may be continuously active, but may wait to collect information when conditions are such that accurate readings are most likely. For example, when one or more accelerometers or gyroscopic components of device 100 indicate that a user is still, at rest, or sleeping, one or more sensors of upper module 110 and/or lower module 150 may collect information from the user while artifacts resulting from physical movement are absent. The accelerometer or gyroscope may not begin reading until the users heart rate measured by another sensor is below a predetermined limit. For example, if the ECG, PPG, or PP demonstrates that the user is moving then the accelerometer or gyroscope may not be turned on. In another example, the accelerometer or gyroscope may turn off if macro-movements are detected or a number of macro-movements are detected above a threshold amount (e.g., 5 or more per min, 10 or more per min, 20 or more per min, 30 or more per min, or 60 or more per minute). Thus the accelerometer or gyroscope may only measure micro-movements if the macro-movements are below the threshold amount (e.g., 20 or less per minute, 10 or less per minute, 5 or less per minute, or 2 or less per minute). Thus, the accelerometer or gyroscope when set, placed, or configured to read micro-movements may only be activated when macro-movements are not present or when macro-movements are infrequent. The accelerometer or gyroscope may measure micro-movements and macro-movements simultaneously and the macro-movements may be considered an outlier and may be removed from reporting.

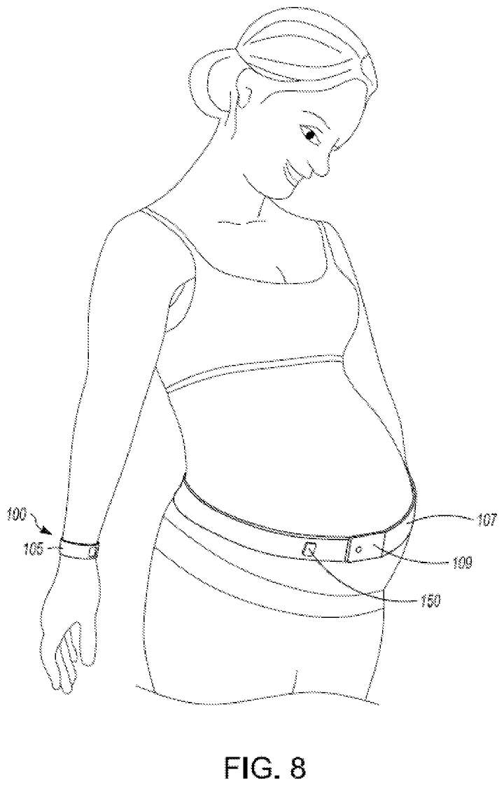

The physiological information from an upper module 110, a lower module 150, or both may be graphically displayed or represented by a waveform. The graphical display may be provided as an output 200. The output 200 may include physiological information of a fetus 212, of a user 214, or both. For example, the information collected may be categorized and then graphically represented as an output or two or more outputs. The one or more outputs may be one or more waveforms, two or more waveforms, or three or more waveforms. The waveforms may be individually created. The waveforms may overlay one another. The waveforms may be created by categorizing the micro-movements. The micro-movements may be categorized by strength of the micro-movements, frequency of the micro-movements, duration of the micro-movements, or a combination thereof. The waveforms may be a one or more waveforms such as a sine wave. FIG. 8 is an output 200 illustrated in a sinusoidal pattern. The output 200 has one graph having both physiological information of a fetus 212 and physiological information of a user 214 located above a graph having only physiological information of a user 214. The physiological information of a fetus 212 is illustrated as a fetal heart rate 202 and the physiological information of a user 214 is a user heart rate 204. The device compares the user heart rate 204 of a lower graph to the user heart rate 204 of the upper graph so that the user heart rate 204 is removed from the fetal heart rate 202. Once the user heart rate 204 or user physiological information of a user 214 is removed from the output 200 physiological information of a fetus 212 or a fetal heart rate 202 can be displayed. As shown, the physiological information of a user 214 is determined with a optical sensor a pulse pressure sensor. The physiological information of a fetus 212 is determined with an accelerometer which may also detect physiological information of a user 214, as shown.

Techniques for estimating a user's blood pressure using pulse signal, pressure, impedance, and other collected and input information has been described above may be employed to estimate a user's oxygen levels (SvO2), hydration, respiration rate, and heart rate variability. For example, PPG, ECG, bio impedance, and acoustic measurements taken from the user can be cross-referenced with the aforementioned library and compared to subjects most closely matching the user (e.g., sex, age, height, weight, race, resting heart rate, BMI, current activity level, and any other medically meaningful distinction). Measured or verified hydration levels of one or more subjects can then be used to estimate the hydration level of the user. A similar process can be employed to estimate the user's oxygen levels (SpO2), respiration rate, and heart rate variability. The ECG measurements can be used to monitor and/or detect heart abnormalities, such as ischemia.

In a portable device, ECG sensors included in the portable device may be augmented with navigation capabilities that can aid a user in making (e.g., taking) ECG measurements or heart rate measurements of a fetus at the proper places of the body (e.g., the chest, belly, mid-section). While not shown in the figures, such as for example FIG. 2, the portable device may include at least one second ECG sensor. The at least one second ECG sensor can be included in one of the lateral sides of the portable device (referred to herein as “side sensors”) so that when the user is holding the portable device between the fingers, at least one of the fingers is in contact with the at least one second ECG sensor. The lateral sides of the portable device are, for example, those sides that are generally perpendicular to the side that includes the first ECG sensor. The portable device may include two accelerometers, gyroscopes, ECG sensors, or a combination thereof so that one may be located on a first side of an abdomen and the second on a second side of the abdomen and the results compared, averaged, verified, calibrated, or a combination thereof.

The portable device may include one side sensor. The portable device includes two side sensors. The two side sensors can be placed on adjacent lateral sides of the portable device. The side sensors can be placed on opposing lateral sides of the portable device. The portable device can include a second sensor (e.g., ECG sensor) that is disposed in a side that is opposite (i.e., referred to herein as the “front sensor”) the side that includes the first sensor (e.g., ECG sensor). Thus, for example, while the user is holding the portable device between users fingers, such as the thumb and the middle finger, the user can place the index finger on the front sensor. The portable device can include the first ECG sensor, at least one side sensor, and a front sensor. The portable device can include the first ECG sensor, at least two side sensors, and a front sensor. In another example, the first sensor and second sensors may be accelerometers and may be located on opposite sides of an abdomen and while the first sensor, the second sensor, or both are taking measurements the user may take a measurement with the ECG sensor.

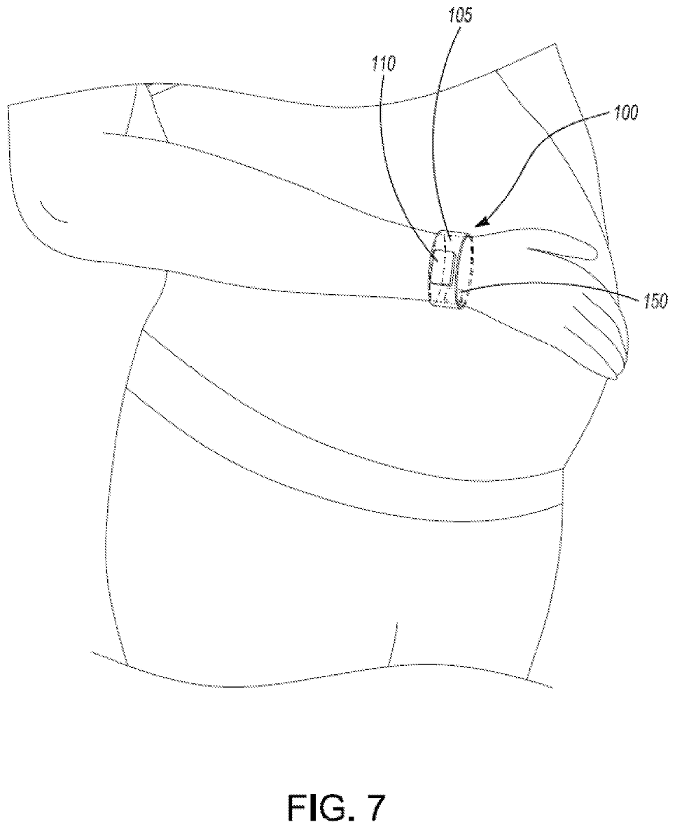

The portable device may be a wrist-worn device (see FIGS. 4B, 4C, 7), which the user can place on the user's chest, mid-section, waist, or a location therebetween while the portable device is worn by the user. The first ECG sensor can be included in a lower module, such as the lower module 150 of FIG. 1. The ECG sensor can be disposed on a side of the lower module that is opposite the side that includes the sensor array 155. The first ECG sensor is included in the side that does not face (e.g., touch, etc.) the wrist of the user. In another example, the portable device may not include a lower module. Thus, the first ECG sensor can be included (e.g., disposed, etc.) in a buckle of the strap of the portable device.

The portable device can include at least one second ECG sensor. The portable device can include a first second ECG sensor in the sensor array 155 and a second second ECG sensor in an upper module, such as the upper module 110 of FIG. 1. Thus, when taking a measurement, the user can place the first ECG sensor on his/her chest, the first second ECG sensor can touch the wrist of the user, and the user can place a finger on the second second ECG sensor.

The portable device can include at least one second ECG sensor (e.g., in a lower module 150) in the strap (e.g., a tail), such as the strap or band 105 of FIG. 3, of the portable device (referred to herein as a strap sensor). For example, the strap sensor may be disposed in a tail of the strap. The tail can be long enough so that the at least one second ECG sensor can be accessible to the user while the wrist of the user is placed on the chest. The user may wear the portable device on a right/left wrist, and as the user places an opposing hand (e.g., the right/left wrist), the user can hold the at least one second ECG sensor between the thumb and index fingers of the other left/right hand. The portable device can be configured to be worn on a wrist of a first arm of the user, the tail of the strap can include a second ECG sensor, and the second ECG sensor can be configured so that the user can hold the second ECG sensor using fingers of the second arm of the user.

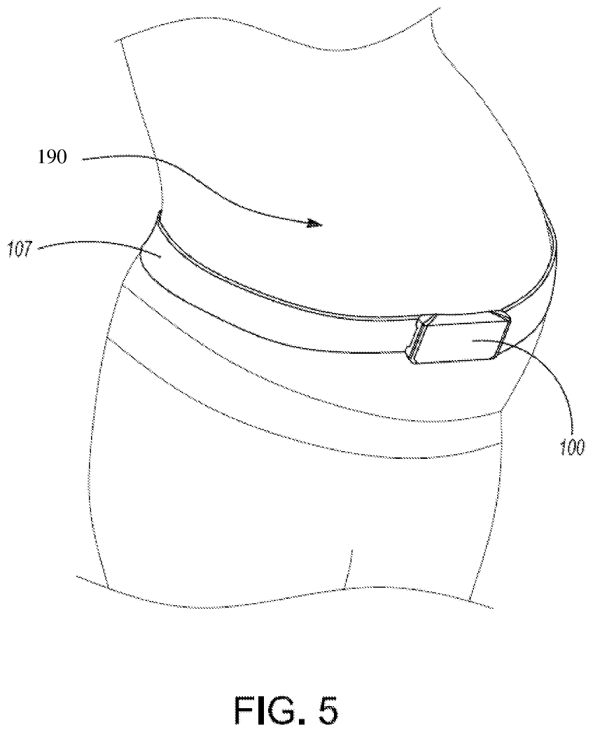

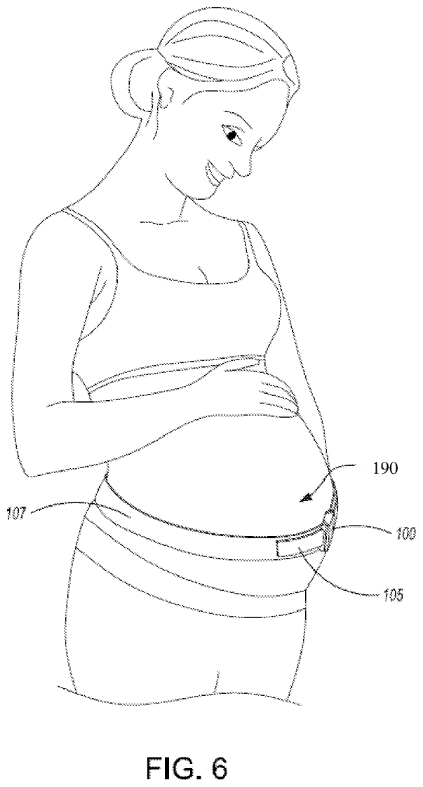

The at least one second ECG sensor or accelerometer can be disposed on the strap of the portable device or in a lower module 150. The at least one second ECG sensor can be disposed on one side of the lower module. The at least one second ECG sensor can be disposed on the strap on both sides of lower module. As such, the at least one second ECG sensor can include at least two strap sensors, which the user can touch while the lower module is placed on the chest or other parts of the user's body. As shown in FIG. 5 the device 100 is located around a mid-section of a user. The device 100 is connected by an extended band 107 that wraps around the mid-section so that both a user heart rate and a fetal heart rate may be measured by the device 100. The extended band 107 may be part of the device 100 and may be expanded to fit a wrist or a mid-section 190 (see FIG. 4B, 4C, or 5). The device 100 may include a band 105 and the device 100 may be removed from a wrist and placed on an extended band 107. The extended band 107 may connect the device 100 to a mid-section 190 of the user so that both the user's heart rate and a fetus heart rate may be monitored (see e.g., FIG. 6). The device 100 may be connected to a user's wrist via a band 105 as is shown in FIG. 7. The device 100 includes an upper module 110 that monitors physiological information of a user and a lower module 150 that monitors physiological information of a fetus within the user by the user placing the device 100 on their mid-section.

A portion of the device 100 may be moved to a second location so that the device 100 is located at both a first location and a second location as is demonstrated in FIG. 8. For example, when only measurement of a user's physiological information is desired the device 100 may be held at a first location and if a fetus's physiological information is desired then the device 100 may be held at a second location. The device 100 includes a band 105 that connects the device to a wrist of a user. The device 100 also includes an extended band 107 with a buckle 109 that connects to the user. As shown, a lower module 150 is removable from the band 105 and connectable to the extended band 107 so that the device 100 monitors the users physiological information at a wrist location and the lower module 150 monitors a fetal physiological information at a mid-section location. The placement (e.g., a current location) of the portable device on the body of the user can be determined based on a displacement from a previous location of the portable device on the body. For example, the upper module 110, the lower module 150, or both may include a displacement device to determine a location or orientation of the device, the upper module 110, the lower module 150, or a combination thereof. The portable device may be previously placed at a first location. That the portable device is currently placed at a second location can be determined by determining (e.g., calculating, measuring, detecting, observing, etc.) the displacement from the first location to the current location.

The displacement can be determined in several ways. In an example, the portable device can include one or more sensors (referred to herein as navigation sensors) that can be used to measure the displacement of the portable device on the body (e.g., chest) of the user. The displacement sensors may determine that the upper module 110, the lower module 150, or both are removed from a band 105 to a second location. To reiterate, data from the navigation sensor can be used to measure the direction and/or length of displacement when the user moves the navigation sensor (e.g., the portable device) along the user's body while or before pressing the portable device to the skin. In another example, the displacement can be determined using a device that is external to the portable device. The location of the portable device may be input by the user, synched from a smart device, requested a move by the portable device based on feedback or programming, or a combination thereof.

The portable device may include navigation sensors. Any number of one or more navigation sensor may be used. Examples of navigation sensors include, but are not limited to, LEDs and photodiodes, an array of photosensors (e.g., minicameras, an optoelectronic sensor) with optional additional light(s), a mechanical sensor with a rolling (e.g., track) ball, or an ultrasound sensor.

For example, a mechanical sensor with a rolling ball, the rolls (e.g., movements, etc.) of the track ball can be converted to an angle and a distance of displacement on the body. For example, with respect to the optoelectronic sensor, successive images of the surface of the chest on which the portable device is moved are taken by the optoelectronic sensor to determine the angle and distance of displacement. Differences between the successive images are used to determine the displacement. For example, in the case of LEDs and photodiodes, several (e.g., 200, more, or fewer) measurements of the white level at one point can be taken and based on changes in the level of luminosity, the displacement can be determined. For example, with respect to an ultrasound sensor, differences between the successive sound reflections are can be to determine the displacement. That is, in the case of an ultrasound transceiver and receiver, several (e.g., 200, more, or fewer) measurements of the sound reflections at one point can be taken, and based on changes in the intensity of reflection (e.g., the echo), the displacement can be determined.

The navigation sensors can be or can include an accelerometer. Accelerometer data can be used to determine a displacement from a previous location to a current location of the portable device on the body of the user. A gyroscope may be additionally be used.

The displacement can be determined using a device that is external to the portable device. The external device can be a device that is in communication with the portable device. The external device can be, for example, a mobile phone or the like, of the user and that is in communication with the portable device. When the user is ready to take ECG measurements, the external device is placed by the user in front of the user so that the external device (e.g., sensors therein) can perceive the location of the portable device on the body of the user.

The portable device and the external device can communicate via wired or a wireless connection. A wired connection can be a Universal Serial Bus (USB) connection, a firewire connection, or the like. A wireless connection can be via a network using Bluetooth communications, infrared communications, near-field communications (NFCs), a cellular data network, or an Internet Protocol (IP) network. In an example, the external device can communicate the location of the portable device on the body of the user to the portable device. The portable device may receive raw sensor data from the external device and the portable device can process the raw sensor data to determine a location of the portable device on the body of the user and/or a displacement of the portable device.

The external device can include a camera, which can be used to take images of the placement of the portable device on the body of the user. Image processing can be used to determine the displacement of the portable device between a first image and a second image. Image processing may be used to determine a current location of the portable device on the body of the user.

The user can be prompted to place the ECG sensor at a reference point of the body. The reference point can be used as the initial reference point for calculating subsequent displacements for identifying the locations of the portable device on the body. The user may be prompted to move the portable device from a first location to a second location (e.g., onto a unique and easily identifiable location on the body, such as above the navel).

The user may be notified with a success signal (e.g., a sound, a haptic tap, a vibration, etc.) that the measurement at the current location is completed. The portable device can notify the user that the measurement was not successful with a failure signal. The measurement may not be successful because, for example, the ECG shape was not recognized in the signal that is received from the first ECG sensor. The ECG shape can be said to be recognized when the ECG shape matches stored normal or ischemic ECG shapes. The portable device may notify the user that the point of the measurement was not recognized.

A system for measuring an electrocardiogram (ECG) of a user includes a portable device that includes a first ECG sensor and an external device that is communication with the portable device. The portable device can include instructions stored in a memory to obtain a first ECG measurement at a first location of a body of the user, identify the first location based on sensor information received from the external device, prompt the user to move the portable device to a second location of the body of the user, and obtain a second ECG measurement at the second location of the body.

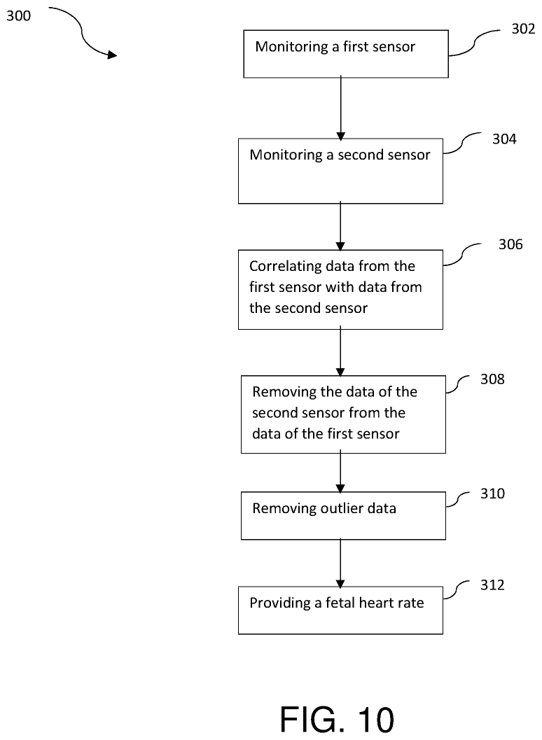

FIG. 10 illustrates a process 300 of determining a fetal heart rate. The process 300 includes a first sensor and monitoring the first sensor 302. The process 300 includes a second sensor and monitoring the second sensor 304. The first sensor and the second sensor may be located at a same location. A processor may receive data from the first sensor, the second sensor, or both. The data from the first sensor and the second sensor may be correlated to one another 306. The process 300 includes a step of removing the data of the second sensor from the data of the first sensor 308. The removed data may isolate a fetal heart rate from a user's heart rate, movements, or both. The process 300 includes a step of removing outlier data 310 that may be caused by macro movements. A fetal heat rate is then provided or displayed 312. The first sensor may be an accelerometer, a gyroscope, or both. The second sensor may be an electrocardiogram (ECG), photoplethysmogram (PPG), or both. The macro movements may be caused by breathing, breathing movements of a fetus, moving of a fetus, movement of the user, or a combination thereof. The first sensor and the second sensor may be located at a same location, on a mid-section of a user, or both. The device may be connected to a band on a user or expanded to fit on a mid-section, a wrist, or both of a user.

It may be appreciated that various changes can be made therein without departing from the spirit and scope of the disclosure. Moreover, the various features of the implementations described herein are not mutually exclusive. Rather any feature of any implementation described herein may be incorporated into any other suitable implementation.

Additional features may also be incorporated into the described systems and methods to improve their functionality. For example, those skilled in the art will recognize that the disclosure can be practiced with a variety of physiological monitoring devices, including but not limited to heart rate and blood pressure monitors, and that various sensor components may be employed. The devices may or may not comprise one or more features to ensure they are water resistant or waterproof. Some implementations of the devices may hermetically sealed.

Other implementations of the aforementioned systems and methods will be apparent to those skilled in the art from consideration of the specification and practice of this disclosure. It is intended that the specification and the aforementioned examples and implementations be considered as illustrative only, with the true scope and spirit of the disclosure being indicated by the following claims.

While the disclosure has been described in connection with certain implementations, it is to be understood that the disclosure is not to be limited to the disclosed implementations but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A device comprising:
an accelerometer and/or gyroscope that is capable of measuring movement;
a sensor that is configured to monitor and determine a electrical signals or pulse signals of a heart so that the sensor detects a heart rate of a user;
wherein the device is configured for placement on a first location of the user to determine a heart rate of the user;
wherein the device is configured for placement on or contact with a second location of the user where the sensor measures the user's heart rate and the accelerometer or gyroscope measures a heart rate of a fetus located within the user; and
a processor configured to isolate the heart rate of the fetus from the heart rate of the user so that the heart rate of the fetus is displayed on the device.

2. The device of claim 1, wherein the device simultaneously measures the heart rate of the fetus and the heart rate of the user.

3. The device of claim 2, wherein the device displays the user's heart rate, the fetus' heart rate, or both.

4. The device of claim 1, wherein the processor analyzes data from the accelerometer or gyroscope and removes outlier information so that macro-movements are eliminated from movement data collected by the accelerometer of gyroscope.

5. The device of claim 4, wherein the macro-movements are movement and/or breathing of the user, the fetus, or both.

6. The device of claim 1, wherein the processor is configured to perform synchrosqueezing to analyze the heart rates so that the processor isolates the heart rate of the fetus from the heart rate of the user.

7. The device of claim 1, wherein the device includes a band that is configured to connect to a wrist of the user and is configured to extend around all or a portion of a mid-section of the user.

8. The device of claim 7, wherein the band is expandable.

9. The device of claim 7, wherein the band is connectable to a longer band that extends completely around the mid-section of the user.

10. A device comprising:
a non-transitory computer-readable medium configured to:
monitor acceleration information from an accelerometer or gyroscope;
monitor electrical signals or pulse signals of a heart with a sensor that is an electrocardiogram (ECG), photoplethysmogram (PPG), or both;
determine a heart rate of a user of the device with the sensor;
determine a heart rate of a fetus of the user with acceleration information from the accelerometer or gyroscope;
determine the heart rate of the fetus by removing the heart rate of the user from data collected so that the heart rate of the fetus is an only remaining data; and
displaying the heart rate of the fetus.

11. The device of claim 10, wherein the non-transitory computer-readable medium is further configured to remove outliers from the acceleration data based upon movement, breathing, or both of the fetus, the user carrying the fetus, or both.

12. The device of claim 11, wherein the movements removed as outliers are macro-movements.

13. The device of claim 10, wherein the device is configured to communicate with a remote device so that the remote device is capable of displaying, recording, or controlling the device and/or data provided by the device.

14. A method comprising:
monitoring an accelerometer or gyroscope to collect acceleration information;
correlating the acceleration information to create a first data set related to a heart rate of a fetus of a user;
monitoring a sensor to determine electrical signals or pulse signals of a heart to create a second data set correlated to the user's heart rate; and
removing the second data set from the first data set so that the user's heat rate is removed from the heart rate of the fetus.

15. The method of claim 14, further comparing: removing any outlier data caused from movement, breathing, or both of a user; movement, breathing, or both of a fetus; or both to determine the heart rate of the fetus.

16. The method of claim 14, further comprising moving a device including the accelerometer or gyroscope and the sensor into contact with or proximate to a second location.

17. The method of claim 14, further comprising monitoring at the second location with the accelerometer or gyroscope and the sensor.

18. The method of claim 14, further comprising expanding a band of a device including the accelerometer or gyroscope and the sensor from a size of a wrist to substantially a size of a mid-section.

19. The method of claim 14, further comprising connecting a device including the accelerometer or gyroscope and the sensor to a band or apparatus spanning all or a portion of a mid-section of a user so that the device is connected to the mid-section.

20. The method of claim 14, wherein the accelerometer or gyroscope are located within a module and the module is removable from a device and the module is connected to a mid-section of a user.
